(19) United States
(12) Patent Application Publication
(10) Pub. No.: US 2007/0065513 A1
(43) Pub. Date: Mar. 22, 2007

(54) Stable lansoprazole formulation

(76) Inventors: Avi Avramoff, Haifa (IL); Valerie Azoulay, Hana (IL)

Correspondence Address: Martin D Moynihan;Prtsi Inc, PO Box 16446, Arlington, VA, 22215, US

(21) Appl. No.: 10/575,809

(22) PCT Filed: Nov. 1, 2004
(86) PCT No.: PCT/US04/32775

§ 371 Date: Apr. 13, 2006

Related U.S. Application Data
(60) Provisional application No. 60/515,672, filed on Oct. 31, 2003.

(51) Int. Cl. A61K 31/4439; A61K 9/26

(52) U.S. Cl. 424/470; 514/338

(57) ABSTRACT

A stable composition comprising a substrate comprising lansoprazole (preferably in the base form), without any alkaline agent; a subcoating layer containing alkaline agent; and an enteric coating layer. The substrate is preferably an inert core with an active layer (containing lansoprazole) layered over it.

### FIELD OF THE INVENTION

The present invention relates to a novel stable formulation for lansoprazole, and methods of preparation and administration thereof, and in particular, for a stable formulation of lansoprazole which is suitable for oral administration and which is efficient to manufacture.

### BACKGROUND OF THE INVENTION

Omeprazole, Pantoprazole, Lansoprazole and other derivatives of benzimidazole, which are active proton pump inhibitors and used conventionally for decreasing gastric secretion are known to be susceptible to degradation and transformation in acid media. Lansoprazole, 2-[[(3-methyl-4-(2,2,2-trifluoroethoxy)-2-pyridyl)methyl]sulfinyl]benzimidazole. Lansoprazole is described for example in U.S. Pat. Nos. 4,628,098, and 4,689,333 and European Patent No. 174726.

Another popular benzimidazole derivative, Omeprazole, 5methoxy-2(((4-methoxy-3,5-dimethyl-2-pyridinyl)methyl)sulfinyl)-1H-benzimidazole, is disclosed and described in European Patent No. 5129 and European Patent No. 124495, as well as in numerous other patents and published patent applications.

The susceptibility of these active proton pump inhibitor substances to degradation and transformation in acid media increases the difficulty of preparing a pharmaceutical form designed for oral administration. If the active substance comes into contact with the stomach content, which is a highly acidic medium, these chemical substances become degraded. Thus, these benzimidazoles should be protected both during storage and during their passage through the acidic environment of the stomach.

The stability of Omeprazole has been extensively studied (see for example A. Pilbrant and C. Cederberg, Scan. J. Gastroenterol, 20: 113-120, 1985). Omeprazole degrades with a half-life of less than 10 minutes in an environment with pH values below 4.0. At pH 6.5 the half life of Omeprazole is 18 hours and at pH 11 about 300 days. Therefore, the environment of Omeprazole should be kept at a sufficiently high pH value in order to maintain the stability of the compound, in a formulation which is suitable as a product for oral administration, for example by locating Omeprazole within a core which also contains alkaline constituents. This leads to an alkaline reaction aimed at improving stability of the active substance during manufacture thereof and duing storage of the pharmaceutical formulation.

In addition, such a formulation must protect Omeprazole from the acidic environment of the stomach, since if Omeprazole is given orally without any protective coating, it will degrade in the acid environment of the stomach. European Patent No. 237,200 discloses one solution, which is to directly coat the solid core containing Omeprazole, or another benzimidazole, with an enteric coating layer.

However, this apparent solution to the instability of Omeprazole caused further complications, in that the alkaline core containing Omeprazole was found to react with the enteric coating, thereby causing the enteric coating to degrade. A solution to these further complications is disclosed in United Kingdom Patent Application No. 2,189,698, in which Omeprazole is contained within a solid active core, which is coated first with a subcoating layer and then with an enteric coating layer. The enteric coating layer protects the Omeprazole during the passage through the stomach, while the subcoating layer protects the enteric coating layer from reacting negatively with the alkaline core containing Omeprazole.

The background art describes other attempts to provide formulations which are suitable for oral administration of acid-labile substances. For example, PCT Application No. WO 97/12581 discloses a composition adapted for oral administration containing Omeprazole which specifically does not include alkaline-reacting compounds. Instead, the composition features a core composed of a nucleus and Omeprazole compressed together, an intermediate layer and an enteric layer.

European Patent No. 519,144 discloses a formulation for Omeprazole, which features a neutral (sugar) core. Omeprazole is sprayed onto the sugar core, after which an intermediate coating layer and an enteric coating layer are sprayed onto the core. Omeprazole is contained in a mixture which features an alkaline reacting substance.

French Application No. 2692,146 discloses stable compositions of microgranules of gastro-protected Omeprazole. The composition features a center of Omeprazole diluted in mannitol. This center is coated with an intermediate layer featuring mannitol. An enteric coating is then added over this intermediate layer. PCT Application No. WO97/12581 discloses a formulation in which an intermediate layer between the core and an enteric coating contains silicium dioxide.

### SUMMARY OF THE INVENTION

The background art does not teach or suggest a formulation for lansoprazole which includes a substrate featuring lansoprazole base but without an alkaline agent, and a subcoating layer that does include an alkaline agent.

The formulation of the present invention contains lansoprazole, preferably in the form of lansoprazole base. The formulation preferably features a substrate comprising lansoprazole (preferably in the base form), without any alkaline agent; a subcoating layer containing alkaline agent; and an enteric coating layer.

Hereinafter, the term “alkaline agent” includes any material which is capable of providing a pH value of at least about 7.0 when present alone in water, preferably at least about 7.5 and more preferably at least about 8.0.

The resultant formulation maintains the stability of lansoprazole during storage and at the same time protects the product during passage through the acidic environment of the stomach, where the acidic environment of the stomach causes a partial ionic exchange to occur within the material of the coating.

The substrate can optionally have several different structures. For example, the substrate is optionally an active core containing lansoprazole (preferably in the base form) but without any alkaline agent, in which the core is a pellet, bead or tablet for example. The active core can be prepared by any conventional method known in the at, including but not limited to, pellets prepared by spheronisation, tablets prepared by granulation and compression, as well as any other methods.

The substrate may also optionally compose an inert core, such as a non pareil seed for example, which is coated with an active layer comprising lansoprazole (preferably in the base form), again without any alkaline agent. The size of the inert core may vary, but preferably lies in the range of from about 80 microns to about 1000 microns, but preferably lies in the range of from about 300 to about 1000 microns.

Optionally and more preferably, the substrate further comprises a cellulosic polymer, including but not limited to, HPMC (hydroxypropyl methylcellulose), HPC (hydroxypropyl cellulose), methylcellulose, carboxymethylcellulose and polyvinylpyrrolidone. HPMC is optionally and preferably Methocel (HPMC E5, which is the grade, relating to the viscosity of HPMC, in this case a low grade; the material is HPMC 2910, which is the substitution type (in this case high substitution). The designation “2910” provides the following information: the first 2 digits, “29”, refer to the approximate percentage content of the methoxy group (OCH3); the second 2 digits, “10”, refer to the approximate percentage content of the hydroxypropoxy group (OCH2CH(OH)CH3), calculated on a dried basis. The type 2910 may be considered to be highly substituted in comparison with two other HPMC polymer variants related to the substitution type (2208 and 2906). HPMC 2910 is a non-limiting example of a suitable material which nay optionally be purchased from Dow Chemicals (USA) or Colorcon (United Kingdom)). Also optionally and more preferably, the substrate further comprises a surfactant such as polysorbate 80 (Tween 80) or sodium lauryl sulfate. Fillers such lactose monohydrate, or any other grade of lactose, may optionally be used.

If the substrate features an active layer on an inert core, then optionally and preferably some type of solvent or solvent mixture is used, more preferably an aqueous solvent such as water for example.

The alkaline agent of the subcoating layer optionally and preferably includes any organic basic salt, including but not limited to sodium stearate. Alternatively or additionally, the alkaline agent may optionally comprise an inorganic basic salt, such as basic inorganic salts of magnesium or calcium, or sodium hydrogen carbonate. Examples of such basic inorganic salts of magnesium include, but are not limited to, heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite [Mg₆Al₂(OH)₁₆.CO₃.4H₂O] and aluminum magnesium hydroxide [2.5MgO.Al₂O₃.xH₂Oπ. Examples of such basic inorganic salts of calcium include, but are not limited to, precipitated calcium carbonate and calcium hydroxide.

The subcoating layer preferably includes any suitable cellulosic polymer, including but not limited to, HPMC (hydroxypropyl methylcellulose), HPC (hydroxypropyl cellulose), methylcellulose, carboxymethylcellulose and polyvinylpyrrolidone. HPMC is optionally and preferably Methocel as previously described.

Also optionally and more preferably, the subcoating layer further comprises a surfactant such as polysorbate 80 (Tween 80) or sodium lauryl sulfate. Fillers such lactose monohydrate, or any other grade of lactose, may optionally be used.

The enteric coating material optionally and preferably includes an enteric material selected from the group consisting of hydroxypropyl methylcellulose phthalate, hydroxypropyl methylcellulose acetate succinate, polyvinyl acetate phthalate, cellulose acetate phthalate, cellulose acetate trimellitate, polymethacrylic acid methyl methacrylate, methacrylic acid copolymers such as Eudragit, preferably Eudragit L30D-55 (poly (methacrylic acid, ethylacrylate), 1:1, dispersion), Eudragit L 100 (poly(methacrylic acid, methylacrylate), 1:1, powder), Eudragit L 100-55 (poly(methacrylic acid, ethylacrylate), 1:1, powder) and Eudragit L12.5 (polymethacrylic acid, methylacrylate 1:1, dispersion).

The enteric coating material of the composition could optionally include a plasticizer. Preferably, the plasticizer is selected from the group consisting of a citric acid ester and a phthalic acid ester.

The enteric coating material could also optionally include a glidant, such as talc or titanium dioxide; and a solvent or a mixture thereof; including but not linked to, an aqueous solvent such as water, or an organic solvent such as isopropyl alcohol or other alcohols, or acetone. Mixtures of aqueous and organic solvents preferably include at least one polar organic solvent such as isopropyl alcohol for example. The enteric coating material could also optionally include a surfactant such as Tween 80 or sodium lauryl sulfate.

According to a first embodiment of the present invention, there is provided a stable composition for lansoprazole, the composition comprising: (a) a substrate, the substrate comprising lansoprazole or a pharmaceutically suitable salt thereof; (b) a subcoating layer for coating the substrate, the subcoating layer comprising an alkaline agent; and (c) an enteric coating material layered over the subcoating layer; wherein the substrate is characterized in that the substrate does not include an alkaline agent.

Optionally, lansoprazole comprises lansoprazole base.

Preferably, the substrate features: (i) a neutral core; and (ii) an active coating containing lansoprazole, the active coating being layered over the neutral core; such that the composition is in a form of a pellet. Optionally, the neutral core comprise a non pareil. Optionally and preferably, the non-pareil has a range in a size of from about 300 to about 1000 microns.

Preferably, the active coating includes at least one cellulosic polymer. More preferably, the at least one polymer is selected from the group consisting of hydroxypropyl methylcellulose (HPMC) and hydroxypropyl cellulose (HPC), or a mixture thereof.

Preferably, the active coating comprises at least one surfactant. More preferably, the at least one surfactant comprises at least one of Tween 80 or sodium lauryl sulfate.

Optionally and preferably, the active coating further comprises at least one filler. More preferably, the at least one filler comprises a suitable grade of lactose.

Optionally, the active coating further comprises an aqueous solvent.

Preferably, the alkaline agent in the subcoating layer comprises an organic basic salt. More preferably, the organic basic salt includes at least one of sodium stearate. Also preferably, the subcoating layer includes at least one cellulosic polymer. More preferably, the at least one polymer is selected from the group consisting of hydroxypropyl methylcellulose (HPMC), ethylcellulose and hydroxypropyl cellulose (HPC), or a mixture thereof.

Preferably, the subcoating layer comprises at least one surfactant. More preferably, the at least one surfactant comprises at least one of Tween 80 or sodium lauryl sulfate.

Preferably, the enteric coating material includes at least one enteric material selected from the group consisting of hydroxypropyl methylcellulose acetate succinate, polyvinyl acetate phthalate, cellulose acetate phthalate, cellulose acetate trimellitate, polymethacrylic acid methyl methacrylate and polymethacrylic acid ethyl methacrylate.

Preferably, the enteric, coating material further comprises a plasticizer. More preferably, the plasticizer is selected from the group consisting of a citric acid ester and a phthalic acid ester.

Optionally and alternatively, the substrate is an active core for containing lansoprazole. Also optionally, the active core is selected from the group consisting of a pellet, a bead and a tablet.

According to another embodiment of the present invention, there is provided a stable composition for lansoprazole, the composition comprising: (a) a substrate, the substrate comprising lansoprazole or a pharmaceutically suitable salt thereof; (b) a subcoating layer for coating the substrate, the subcoating layer consisting essentially of an alkaline agent, a cellulosic polymer, a filler, a surfactant and a solvent; and (c) an enteric coating material layered over the subcoating layer.

According to still another embodiment of the present invention, there is provided a method for administering a therapeutically effective amount of lansoprazole to a subject comprising: administering orally to the subject a stable composition for lansoprazole comprising: (a) a substrate, the substrate comprising lansoprazole or a pharmaceutically suitable salt thereof; (b) a subcoating layer for coating the substrate, the subcoating layer consisting essentially of an alkaline agent, a cellulosic polymer, a filler, a surfactant and a solvent; and (c) an enteric coating material layered over the subcoating layer.

According to yet another embodiment of the present invention, there is provided a method for administering a therapeutically effective amount of lansoprazole to a subject comprising: administering orally to the subject a stable composition for lansoprazole comprising: (a) a substrate, the substrate comprising lansoprazole or a pharmaceutically suitable salt thereof; (b) a subcoating layer for coating the substrate, the subcoating layer comprising an alkaline agent; and (c) an enteric coating material layered over the subcoating layer; wherein the substrate is characterized in that the substrate does not include an alkaline agent.

For the method according to the present invention, the formulation according to the present invention may optionally be determined according to any of the embodiments and implementations described herein.

As used herein, the term “lansoprazole” preferably refers to lansoprazole base, but may optionally refer to one of its single enantiomers or an alkaline salt of lansoprazole or one of its single enantiomers.

### DESCRIPTION OF THE PREFERRED EMBODIMENTS

The formulation of the present invention contains lansoprazole, preferably in the form of lansoprazole base. The formulation preferably features a substrate comprising lansoprazole (preferably in the base form), without any alkaline agent; a subcoating layer containing alkaline agent; and an enteric coating layer.

As shown by the in vitro data given below, the formulation of the present invention has been shown to be particularly effective for the oral administration of lansoprazole, a result which could not have been predicted from then references.

The preparation of the compositions of the present invention is described first with reference to the following general description and then with reference to the following non-limiting examples of the preparation and application of the compositions of the present invention.

As noted previously, the formulation of the present invention includes a substrate which features lansoprazole. The substrate is preferably prepared by dissolving lansoprazole in an aqueous dispersion, optionally also including at least one filler, at least one cellulosic polymer and at least one surfactant. This solution is then sprayed over an inert core. Alternatively, the substrate may optionally be prepared without an inert core, by compression or wet granulation of these ingredients, or extrusion and spheronisation, or through any other suitable preparation method thereof.

The subcoating layer is then coated over the substrate. Preferably, the subcoating layer is prepared by adding an organic basic salt, more preferably sodium stearate, as the alkaline agent, to an aqueous solution. Alternatively, the alkaline agent could be an inorganic basic salt as described below. The solution may also optionally include other ingredients, such as one or more surfactants, and/or one or more fillers, and/or one or more cellulosic polymers.

A solution is then prepared with the enteric coating material. The solution preferably includes a solvent or a mixture thereof, including but not limited to, an aqueous solvent such as water, or an organic solvent such as isopropyl alcohol or other alcohols such as ethanol, or acetone. Mixtures of aqueous pad organic solvents preferably include at least one polar organic solvent such as isopropyl alcohol for example. The solution may also optionally and preferably include a plasticizer, and/or a glidant and/or a surfactant.

This enteric coating solution is then layered over the previously coated (with the subcoating material) substrate to form the composition of the present invention.

The term “substrate” refers to substantially any structure which features lansoprazole. Preferably, lansoprazole is in the form of lansoprazole base. The amount of lansoprazole optionally and preferably ranges from about 2% to about 30% over the total formulation, weight per weight of the base. For example, this structure could be an active core containing the lansoprazole. This active core could be prepared in a number of different ways which are known in the art. For example, the active core could be formed by compressing lansoprazole with the additional ingredient(s). As another example, the active core could be prepared by mixing lansoprazole with the additional ingredient(s), spheronizing the mixture and then forming cores through pelletisation. The active core is also optionally formed by granulating the active ingredient with the additional ingredient(s) and compressing the granulation into tablets. The active core is also optionally formed by preparing pellets as previously described, and then compressing the pellets into a tablet.

Alternatively and optionally, the structure could include a neutral core, such as a sugar bead which does not contain lansoprazole, over which lansoprazole is coated. The coating includes lansoprazole with a suitable adhesive polymer. For example, optionally and preferably, the active coating includes from about 0.1% to about 2% surfactant; from about 2% to about 10% of lactose monohydrate or any other grade of lactose; from about 2% to about 10% of a cellulosic polymer, preferably HPMC; and a solvent, such as water for example.

The subcoating layer preferably includes a cellulosic polymer and an alkaline agent. The alkaline agent may optionally include a basic orgainc salt or a basic inorganic salt, preferably in an amount of from about 0.1% to about 10%, weight per weight over the formulation. Examples of basic organic salts include but are not limited to any one or more of sodium stearate. Alternatively or additionally, the alkaline agent may optionally comprise an inorganic basic salt, such as basic inorganic salts of magnesium or calcium, or sodium hydrogen carbonate. Examples of such basic inorganic salts of magnesium include, but are not limited to, heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite [Mg₆Al₂(OH)₁₆.CO₃.4H₂O] and aluminum magnesium hydroxide [2.5MgO.Al₂O₃.xH₂O]. Examples of such basic inorganic salts of calcium include, but are not limited to, precipitated calcium carbonate and calcium hydroxide.

The cellulosic polymer optionally and preferably includes any one or more of HPMC (hydroxypropyl methyl cellulose), HPC (hydroxypropyl cellulose), methylcellulose, carboxymethylcellulose and polyvinylpyrrolidone. HPMC is optionally a preferably Methocel. The cellulosic polymer is optionally and preferably present in an amount of from about 2% to about 10%.

Also optionally and more preferably, the subcoating layer further comprises a surfactant such as polysorbate 80 (Tween 80) or sodium lauryl sulfate, most preferably in an amount of from about 0.1% to about 2%. Fillers such lactose monohydrate, or any other grade of lactose, may optonally be used.

Substantially any type of suitable enteric coating material could be used in order to cost the substrate, including but not limited to, cellulose acetate phthalate (CAP); hydroxypropyl methylcellulose phthalate (HPMCP); polyvinyl acetate phthalate; cellulose acetate trimellitate; polymethacrylic acid methyl methacrylate or ethyl methacrylate, such as the various types of Eudragit; and hydroxypropyl methylcellulose acetate succinate (HPMCAS). The concentration range of the enteric coating material is preferably in a range of from about 5% to about 30% weight per weight over the entire formulation.

The enteric coating optionally contains a plasticizer, such as a citric acid ester, a phthalic acid ester, or any suitable plasticizer.

The method for applying the subcoating material and/or the enteric coating material to the substrate can vary. Substantially any coating method can be used, such as pan coating or fluidized bed coating, with the solution of the enteric coat chosen.

The following specific examples illustrate various aspects of the compositions of the present invention, and are not intended to be limiting in any way. Specific reference is made to lansoprazole for the purposes of description only and without intending to be limiting.

### EXAMPLE 1

This example of the composition of the present invention was prepared as follows. Inert cores (sugar spheres or non pareils) of size from about 710 to about 850 microns were used. The active layer contained lansoprazole; polysorbate 80 (Tween 80) as the surfactant; lactose monohydrate; Methocel (HPMC E5) and water as the solvent.

The subcoating layer included sodium stearate as the alkaline agent; lactose monohydrate as the filler, HPMC E5; Tween 80 as the surfactant; and water as the solvent.

The enteric coating layer included Eudragit 1100-55 (methacrylic acid copolymer c) as the enteric polymer; triethyl citrate as the plasticizer; tale as the glidant; and a mixture of isopropyl alcohol and water as the solvent.

TABLE 1
Substrate (Inert Core with Active Layer)

| Ingredients | Quantity per tablet |
|---|---|
| Non-pareil sugar beads (inert core) | 110 mg |
| Lansoprazole | 30 mg |
| Tween 80 | 5 mg |
| Lactose monohydrate | 25 mg |
| HPMC E5 | 25 mg |
| Water | not present in the final |
| | formulation, as the |
| | formulation is dried |

TABLE 2
Subcoating layer

| | |
|---|---|
| sodium stearate | 2 mg |
| Lactose monohydrate | 25 mg |
| HPMC E5 | 25 mg |
| Tween 80 | 5 mg |
| Water | not present in final formulation because |
| | of drying; used only as a solvent |

Tables Enteric coating layer

| | |
|---|---|
| Eudragit L100-55 | 45 mg |
| triethyl citrate | 6 mg |
| Talc | 23 mg |
| isopropyl alcohol | not present in final formulation because of |
| | drying; used only as a solvent |
| Water | not present in final formulation because of |
| | drying; used only as a solvent |

The above illustrative formulation was prepared according to the following process. It should be noted that this process is intended as an example only and is not meant to be limiting in any way.

First sugar spheres (non-pareil sugar beads) were placed in a tangential spray fluid bed coater. Next, the active layer coating ingredients were prepared as a suspension in water such that the total concentration of solids in water was approximately 18%. This suspension was prepared by dissolving HPMC E5 in a portion of the water (approximately 60% of the total water used), aftr which Tween 80, lactose monohydate and lansoprazole (active ingredient) were suspended in the remaining portion of water. These two suspension preparations were then mixed together to form the active coating suspension.

The active coating suspension was spayed onto the sugar beads, thereby forming the substrate. A suspension of the subcoating layer was then prepared, so that the concentration was approximately 11% of the total solids in water. The subcoating (intermediate) layer suspension was prepared by again first dissolving HPMC E5 in a portion of the water (about 50% of the total water used), afer which Tween 80 and lactose monohydrate were suspended in the remaining portion of water. These two suspension preparations were then mixed together to form the subcoating suspension.

The substrate was then coated with the subcoating suspension to form a coated substrate. An enteric coating layer dispersion was then prepared as follows. Isopropyl alcohol and water were first mixed together, after which triethyl citrate was dissolved into the mixture. Eudragit L100-55 was then added and dissolved into the mixture, followed by talc. The enteric coating dispersion was layered over the coated substrate to form the finished pellets. The pellets were then filled into capsules.

### EXAMPLES 2

This example features the same formulation as Example 1 but the sugar spheres are much smaller (500-600 microns). A similar method of preparation was followed as for Example 1

### EXAMPLE 3

This example features the same formulation as Example 1 for the substrate and subcoating layer. The enteric coating is different and preferably includes HPMC acetate succinate and acetone as the solvent.

TABLE 4
Enteric coating layer

| | |
|---|---|
| HPMC acetate succinate | 74 mg |
| acetone | not present in final formulation because |
| | of drying; used only as a solvent |

The composition was prepared as for the illustrative process of Example 1, with regard to preparing the coated substrate (coated with the subcoating layer). The composition was prepared in a fluid bed coating chamber, equipped with a Wurster bottom-spraying device. An enteric dispersion was then prepared as follows. The HPMC acetate succinate was dissolved in acetone in a concentration of 10%. The enteric coating was layered over the subcoated pellets in order to form the finished pellets. The pellets were then filled into capsules.

### EXAMPLE 4

This example features the same formulation as Example 3 but the sugar spheres are much smaller (500-600 microns).

A similar method of preparation was followed as for Example 3.

### EXAMPLE 5

The example is similar to the formulation of Example 1 for the substrate and the subcoating layer. The enteric coating layer is different and preferably includes HPMC acetate succinate and a plasticizer, with water as the solvent.

TABLE 5
Enteric coating layer

| | |
|---|---|
| HPMC acetate succinate | 40 mg |
| Triethyl citrate (plasticizer) | 11.5 mg |
| Sodium lauryl sulfate | 1.2 mg |
| Talc | 20 mg |
| Water | not present in final formulation |
| | because of drying; used only as a |
| | solvent |

The composition was prepared as for the illustrative process of Example 1, with regard to preparing the coated substrate (coated with the subcoating layer). The composition was prepared in a fluid bed coating chamber, equipped with a Wurster bottom-spraying device. An enteric dispersion was then prepared as follows. Triethyl citrate and sodium lauryl sulfate were dissolved in water. HPMC acetate succinate was then added to the solution to form a dispersion. Talc was finally added to the dispersion. The enteric coating was layered over the subcoated pellets in order to form the finished pellets. The pellets were then filled into capsules.

### EXAMPLE 6

Stability tests were performed with formulations prepared according to Examples 1-3. For all tests, capsules were filed with coated pellets prepared according to these Examples. These filled capsules were then packed into an Alu/Alu (Aluminum/Aluminum) blister, which is a well known technique in the art for packing certain oral dosage forms. The blister was then stored under accelerated conditions of 30° C. and 60% relative humidity; or 40° C. and 75% relative. Samples of the capsules were examined initially, and after one month of storage under one of these conditions. In addition, samples were assayed to determine the amount of lansoprazole present in the capsule, as listed under “Assay” as milligrams of lansoprazole per capsule. A dissolution test was performed, using the accepted USP method. The capsules were placed in 0.1 N HCl for 1 hours, followed by a solution at pH 6.8 with stirring with a paddle at 75 rpm for 60 minutes. Gastric resistance was also examined by placing the capsules in a simulated gastric fluid for 2 hours (pH of approximately 1), as is well known in the art. The results are shown in the table below.

TABLE 6A
Results of stability tests

| | | | | |
|---|---|---|---|---|
| TEST | REQUIRED | | | |
| PERFORMED | RESULT | EXAMPLE 1 | EXAMPLE 2 | EXAMPLE 3 |

INITIAL RESULTS AT START OF TEST

| | | | | |
|---|---|---|---|---|
| Appearance | White to off | Conform | Conform | Conform |
| | white pellets | | | |
| Assay | 95-105% | 103% | 103% | 103% |
| | (amount of active | | | |
| | ingredient) | | | |
| Gastric resistance | NLT (not less | 103% | 98% | 98% |
| | than) 85% | | | |
| Dissolution | NLT 80% | 105% | 106% | 103% |
| Known | NMT (not more | 0.19% | 0.19% | 0.19% |
| individual | than) 0.5% | | | |
| impurity | | | | |
| Unknown | NMT 0.2% | 0.08% | 0.08% | 0.08% |
| individual | | | | |
| impurity | | | | |
| Total impurity | NMT 1% | 0.33% | 0.33% | 0.34% |

1 MONTH 30 DEGREES, 60% RH (relative humidity)

| | | | | |
|---|---|---|---|---|
| Appearance | White to off | Conform | Conform | Conform |
| | white pellets | | | |
| Assay | 95-105% | 99.5% | 98.1% | 97.3% |
| Gastric resistance | NLT 85% | 103% | 103% | 96% |
| Dissolution | NLT 80% | 106% | 105% | 100% |
| Known | NMT 0.5% | 0.13% | 0.13% | 0.11% |
| individual | | | | |
| impurity | | | | |
| Unknown | NMT 0.2% | 0.11% | 0.06% | 0.07% |
| individual | | | | |
| impurity | | | | |
| Total impurity | NMT 1% | 0.38% | 0.32% | 0.23% |

TABLE 6B
ADDITIONAL RESULTS - STABILITY OF EXAMPLE 3 (9 MONTHS)

| | | | | 6 | 6 | 9 | 9 |
|---|---|---|---|---|---|---|---|
| | | 3 months | 3 months | months | months | months | months |
| | | RT/ | 30° C./ | RT/ | 30° C./ | RT/ | 30° C./ |
| | Spec. | 60% RH | 60% RH | 60% RH | 60% RH | 60% RH | 60% RH |
| Appearance | White to off | Conform | Conform | Conform | Conform | Conform | Conform |
| | white pellets | | | | | | |
| Assay | 95-105% | 101% | 102% | 101% | 101% | 100% | 98% |
| Gastric | NLT 85% | 99% | 97% | 96% | 97% | 96% | 93% |
| resistance | | | | | | | |
| Dissolution | NLT 80% | 99% | 103% | 102% | 101% | 102% | 102% |
| Known | NMT 0.5% | 0.15% | 0.15% | 0.21% | 0.26% | 0.15% | 0.17% |
| individual | | | | | | | |
| impurity | | | | | | | |
| Unknown | NMT 0.2% | 0.06% | 0.13% | 0.11% | 0.1% | 0.06% | 0.15% |
| individual | | | | | | | |
| impurity | | | | | | | |
| Total impurity | NMT 1% | 0.37% | 0.52% | 0.53% | 0.81% | 0.62% | 0.96% |

These results show that the capsules, prepared according to Examples 1-3, show good stability and gastric resistance, yet are also able to dissolve in an appropriate time-dependent manner.

### EXAMPLE 7

### Method of Administration

The formulation of the present invention may optionally be administered to a subject, optionally for any suitable use for lansoprazole as a treatment (for example to treat any condition for which treatment with lansoprazole is suitable). Dosing regimens, including amount of each dose and dosing frequency, may easily be determined by one of ordinary skill in the art as such regimens are well known for lansoprazole.

The method according to the present invention for administering a therapeutically effective amount of lansoprazole to a subject preferably includes administering orally to the subject a stable composition for lansoprazole comprising a formulation according to the present invention.

### EXAMPLE 8

### Additional Formulation

This example features the same formulation as Example 3 except that the sugar spheres (non-pareils) are much smaller (200-300 microns). It should be noted that using smaller beads or spheres is more suitable for compression to a Multiple Unit formulation (described below). A particularly preferred size range for such compression is from about 200 to about 300 microns.

A similar method of preparation was followed as for Example 3.

### EXAMPLE 9

### In vivo Bioavailability Study

A two-way bioavailability study was performed for testing the pharmacokinetic profile of exemplary capsules according to the present invention, which were prepared according to the formulation described in Example 1. The study was performed with ten healthy male volunteers, who received the test formulation prepared according to Example 1 in comparison to the reference product, which is the 30 mg Lansoprazole dosage form of the formulation of Wyeth. The study was conducted as described below with regard to Example 10.

Comparable bioavailability was achieved with the capsules of the present invention, relative to values obtained with the reference product. Furthermore, the values of Cmax and AUC concerning the rate of absorption for the capsules of the present invention were comparable to results obtained for the reference.

TABLE 7
Bioavailabilitv

| | AUC | Cmax |
|---|---|---|
| | (ng × hour/ml) | (ng/ml) |
| Formulation according to | 1790.18 +/− 1247.19 | 676.15 +/− 288.53 |
| the present invention | (476.9; 4168.6) | (230.6; 1088.7) |
| Reference product | 1813.80 +/− 1028.66 | 716.06 +/− 168.47 |
| | (845.1; 4098.4) | (433.9; 934.5) |
| Ratio* | 0.91 | 0.88 |

*The presented ratios are geometric means of the individual ratios between test and reference parameters. Parametric estimators with logarithmic transformation are used.

Thus, the capsules of the present invention clearly show good performance both in vitro, as described in Example 6, and in vivo.

### EXAMPLE 10

### Expanded in vivo Bioavailability Study

The formulation prepared according to Example 3 above was tested for bioavailability in vivo by administration to 50 human subjects, in an expanded bioavailability study. Briefly, the results showed clear bioequivalence between the formulation according to the present invention and the reference product.

A bioequivalence study was performed in order to assess the relative bioavailability of the test product (capsules prepared according to Example 3) in comparison to the reference product ZOTON 30 mg capsules (Wyeth) after a single dose administration. The study was designed as monocentric, open, randomized, single dose, two-way crossover study in healthy volunteers with a wash-out period of one week between the last dose in period 1 and the first dose in period 2, such that each volunteer served as his own control. Fifty healthy, male volunteers were planned for and concluded the study.

At each period, 1 capsule of either formulation was administered once to fasting volunteers. Blood samples were withdrawn before the administration and at the following times: 0.25; 0.5; 0.75; 1; 1.25; 1.50; 1.75; 2; 2.50; 3; 3.50; 4; 5; 6; 9; and 12 hours after the dose was administered.

Plasma concentrations of lansoprazole were determined using HPLC analytical method with UV detection.

TABLE 8A
PHARMACOKINETIC PARAMETERS

| | AUC(0-∞) |
|---|---|
| | (ng × hour/ml) |
| Formulation according to | 1946.91 +/− 2232.50 |
| the present invention | (517.72; 11020.42) |
| Reference product | 1844.94 +/− 2065.38 |
| | (449.23; 10094.23) |
| CV % | 25% |
| (Coefficient of Variation) | |
| RATIO* | 1.07 |
| (90% ANOVA C.I.) | (0.96; 1.06) |

TABLE 8B
PHARMACOKINETIC PARAMETERS

| | Tmax |
|---|---|
| | (hours) |
| Formulation according to | 2.30 +/− 0.85 |
| the present invention | (1.00; 5.00) |
| Reference product | 1.70 +/− 1.00 |
| | (0.50; 5.00) |
| DIFFERENCE | 0.63 |
| ESTIMATE** | (−3.00; 3.75) |
| (range) | (0.38; 0.88) |
| (90% non parametric C.I.) | |

The presented values for all pharmacokinetic parameters are mean ± SD and (range).
* The presented ratios are the geometric means of the ratios between test and the reference parameters. Parametric estimators and Parametric Confidence Intervals, based on the linear model with logarithmic transformation (multiplicative model), are brought.
**The presented difference is the median difference with its corresponding range. 90% non-parametric Confidence Intervals for the median difference with its corresponding median estimate was computed by the method of Hauschke et al., which does not require the restrictive assumption of equal period effect as previous methods.

### EXAMPLE 11

### Multiple Unit Formulations

The formulations prepared according to the present invention may optionally be prepared as a Multiple Unit formulation. A Multiple Unit formulation is a pharmaceutical multiple unit tableted dosage form, in which the active substance is in the form of individually enteric coating layered units (preferably pellets as described below, but optionally including small beads, particles or granules) compressed into a tablet. The enteric coating layer(s) covering the individual units of active substance has properties such that the compression of the units into a tablet does not significantly affect the acid resistance of the individually enteric coating layered units. The active substance, lansoprazole, is therefore protected from degradation and dissolution in acidic media and has a good stability during long-term storage.

As previously described, the Multiple Unit formulation may optionally be prepared according to any of the above Examples with a neutral core; optionally and preferably, the non-pareil (sugar bead) used for the neutral core has a range in a size of from about 80 to about 1000 microns.

The Multiple Unit formulation preferably features lansoprazole as an active ingredient. The formulation also preferably features a substrate which includes lansoprazole or a pharmaceutically suitable salt thereof. The substrate is preferably covered by a subcoating layer which includes an alkaline agent. An enteric coating material is then layered over the subcoating layer to form enteric coated pellets. Therefore, the enteric coated pellets may optionally be prepared according to any of the formulations and methods described above. Next, the enteric coated pellets are compressed into a tablet dosage form, to form the Multiple Unit formulation.

Preferably, the substrate features a neutral core; and an active coating containing lansoprazole, in which the active coating is layered over the neutral core, such that the composition is in a form of a pellet. The neutral core preferably comprises a sugar bead (non-pareil), with a size in the range of from about 80 to about 1000 microns, more preferably in the range of from about 80 to about 500 microns.

Optionally and preferably, the enteric coating does not include a plasticizer for better compression properties and/or properties of the coating.

While the invention has been described with respect to a limited number of embodiments, it will be appreciated that many variations, modifications and other applications of the invention may be made.

1. A stable composition for lansoprazole, the composition comprising:
(a) a substrate, said substrate comprising lansoprazole or a pharmaceutically suitable salt thereof;
(b) a subcoating layer comprising an alkaline agent; and
(c) an enteric coating material layered over said subcoating layer; wherein said substrate is characterized in that said substrate does not include an alkaline agent.

2. The composition of claim 1, wherein lansoprazole comprises lansoprazole base.

3. The composition of claim 1, wherein said substrate features:
(i) a neutral core; and
(ii) an active coating containing lansoprazole, said active coating being layered over said neutral core;
such that the composition is in a form of a pellet.

4. The composition of claim 3, wherein said neutral core comprises a non pareil.

5. The composition of claim 4, wherein said non-pareil has a range in a size of from about 300 to about 1000 microns.

6. The composition of claim 3, wherein said active coating includes at least one cellulosic polymer selected from the group consisting of hydroxypropyl methylcellulose (HPMC) and hydroxypropyl cellulose (HPC), or a mixture thereof.

7. (canceled)

8. The composition of claim 3, wherein said active coating comprises at least one surfactant selected from the group consisting of of Tween 80 or sodium lauryl sulfate.

9. (canceled)

10. The composition of claim 3, wherein said active coating further comprises at least one filler.

11. The composition of claim 10, wherein said at least one filler comprises a suitable grade of lactose.

12. The composition of claim 3, wherein said active coating further comprises an aqueous solvent.

13. The composition of claim 1, wherein said alkaline agent in said subcoating layer comprises an organic basic salt.

14. The composition of claim 13, wherein said organic basic salt includes at least one of sodium stearate.

15. The composition of claim 1, wherein said alkaline agent in said subcoating layer comprises an inorganic basic salt.

16. The composition of claim 1, wherein said subcoating layer includes at least one cellulosic polymer selected from the group consisting of hydroxypropyl methylcellulose (HPMC), ethylcellulose and hydroxypropyl cellulose (HPC), or a mixture thereof.

17. (canceled)

18. The composition of claim 1, wherein said subcoating layer comprises at least one surfactant selected from the group consisting of Tween 80 or sodium lauryl sulfate.

19. (canceled)

20. The composition of claim 1, wherein said enteric coating material includes at least one enteric material selected from the group consisting of hydroxypropyl methylcellulose acetate succinate, polyvinyl acetate phthalate, cellulose acetate phthalate, cellulose acetate trimellitate, polymethacrylic acid methyl methacrylate and polymethacrylic acid ethyl methacrylate.

21. The composition of claim 1, wherein said enteric coating material further comprises a plasticizer selected from the group consisting of a citric acid ester and a phthalic acid ester.

22. (canceled)

23. The composition of claim 1, wherein said substrate is an active core for containing lansoprazole.

24. The composition of claim 23, wherein said active core is selected from the group consisting of a pellet, a bead and a tablet.

25. A stable composition for lansoprazole, the composition comprising:
(a) a substrate, said substrate comprising lansoprazole or a pharmaceutically suitable salt thereof;
(b) a subcoating layer for coating said substrate, said subcoating layer consisting essentially of an alkaline agent, a cellulosic polymer, a filler, a surfactant and a solvent; and
(c) an enteric coating material layered over said subcoating layer.

26. A method for administering a therapeutically effective amount of lansoprazole to a subject comprising:
administering orally to the subject a stable composition for lansoprazole comprising:
(a) a substrate, said substrate comprising lansoprazole or a pharmaceutically suitable salt thereof;
(b) a subcoating layer for coating said substrate, said subcoating layer consisting essentially of an alkaline agent, a cellulosic polymer, a filler, a surfactant and a solvent; and
(c) an enteric coating material layered over said subcoating layer.

27. A method for administering a therapeutically effective amount of lansoprazole to a subject comprising:
administering orally to the subject a stable composition for lansoprazole comprising:
(a) a substrate, said substrate comprising lansoprazole or a pharmaceutically suitable salt thereof;
(b) a subcoating layer for coating said substrate, said subcoating layer comprising an alkaline agent; and
(c) an enteric coating material layered over said subcoating layer;
wherein said substrate is characterized in that said substrate does not include an alkaline agent.

28. The method of claim 27, wherein lansoprazole comprises lansoprazole base.

29. The method of claim 27, wherein said substrate features:
(i) a neutral core; and
(ii) an active coating containing lansoprazole, said active coating being layered over said neutral core;
such that the composition is in a form of a pellet.

30. The method of claim 29, wherein said neutral core comprises a non pareil.

31. The method of claim 30, wherein said non-pareil has a range in a size of from about 300 to about 1000 microns.

32. The method of claim 29, wherein said active coating includes at least one cellulosic polymer selected from the group consisting of hydroxypropyl methylcellulose (HPMC) and hydroxypropyl cellulose (HPC), or a mixture thereof.

33. (canceled)

34. The method of claim 29, wherein said active coating comprises at least one surfactant selected from the group consisting of Tween 80 or sodium laurel sulfate.

35. (canceled)

36. The method of claim 29, wherein said active coating further comprises at least one filler.

37. The method of claim 36, wherein said at least one filler comprises a suitable grade of lactose.

38. The method of claim 29, wherein said active coating further comprises an aqueous solvent.

39. The method of claim 27, wherein said alkaline agent in said subcoating layer comprises an organic basic salt.

40. The method of claim 39, wherein said organic basic salt includes at least one of sodium stearate.

41. The method of claim 27, wherein said alkaline agent in said subcoating layer comprises an inorganic basic salt.

42. The method of claim 27, wherein said subcoating layer includes at least one cellulosic polymer selected from the group consisting of hydroxypropyl methylcellulose (HPMC), ethylcellulose and hydroxypropyl cellulose (HPC), or a mixture thereof.

43. (canceled)

44. The method of claim 27, wherein said active coating comprises at least one surfactant selected from the group consisting of Tween 80 or sodium lauryl sulfate.

45. (canceled)

46. The method of claim 27, wherein said enteric coating material includes at least one enteric material selected from the group consisting of hydroxypropyl methylcellulose acetate succinate, polyvinyl acetate phthalate, cellulose acetate phthalate, cellulose acetate trimellitate, polymethacrylic acid methyl methacrylate and polymethacrylic acid ethyl methacrylate.

47. The method of claim 27, wherein said enteric coating material further comprises a plasticizer selected from the group consisting of a citric acid ester and a phthalic acid ester.

48. (canceled)

49. The method of claim 27, wherein said substrate is an active core for containing lansoprazole.

50. The method of claim 49, wherein said active core is selected from the group consisting of a pellet, a bead and a tablet.

51. A stable composition for lansoprazole, the composition comprising:
(a) a neutral core; and
(b) an active coating containing lansoprazole base, said active coating being layered over said neutral core to form a coated core;
(c) a subcoating layer for coating said coated core, said subcoating layer comprising an alkaline agent; and
(d) an enteric coating material layered over said subcoating layer;
wherein said active coating is characterized in that said active coating does not include an alkaline agent and such that the composition is in a form of a pellet.

52. The composition of claim 2, wherein said neutral core has a size in a range of from about 80 to about 1000 microns.

53. A stable composition for Lansoprazole, the composition comprising:
(a) a substrate, said substrate comprising lansoprazole or a pharmaceutically suitable salt thereof;
(b) a subcoating layer comprising an alkaline agent;
(c) an enteric coating material layered over said subcoating layer to form enteric coated pellets;
wherein said enteric coated pellets are compressed into a tablet dosage form.

54. The composition of claim 53, wherein said substrate features:
i) a neutral core; and
ii) an active coating containing lansoprazole, said active coating being layered over said neutral core;
such that the composition is in a form of a pellet.

55. The composition of claim 54, wherein said neutral core has a size in a range of from about 80 to about 500 microns.

56. The composition of claim 55, wherein said size is in a range of from about 200 to about 300 microns.

57. The composition of claim 53, wherein said enteric coating does not include a plasticizer.
